SEQUENCE LISTING (6 sequences)

SEQ ID NO: 1 - DNA, 41 bp, Artificial Sequence
  synthetic construct; primer
gcggtctgggtaccttcgtaccggtcacgagttcgaaatcg

SEQ ID NO: 2 - DNA, 42 bp, Artificial Sequence
  synthetic construct; primer
agttaccaaaggtggtccgctggctttggaaccgtactggaa

SEQ ID NO: 3 - DNA, 20 bp, Artificial Sequence
  synthetic construct; primer
gttcgtatggaaggttccgt

SEQ ID NO: 4 - DNA, 18 bp, Artificial Sequence
  synthetic construct; primer
cagccgggtgtttaacgt

SEQ ID NO: 5 - DNA, 20 bp, Artificial Sequence
  synthetic construct; primer
ccacttccacttccagcagg

SEQ ID NO: 6 - DNA, 21 bp, Artificial Sequence
  synthetic construct; primer
cgttcgcttgggacatcctgt

(19) United States
(12) Patent Application Publication
(10) Pub. No.: US 2023/0054038 A1
(43) Pub. Date: Feb. 23, 2023

(54) DEVICES, SYSTEM AND METHODS FOR TRACKING PRODUCTS USING BIOLOGICAL BARCODES AND GENETICALLY MODIFIED ORGANISMS CONTAINING THE SAME

(71) Applicant: Aanika Biosciences, Inc., Brooklyn, NY (US)

(72) Inventors: Vishaal Bhuyan, Brooklyn, NY (US); Eduardo Morales, Brooklyn, NY (US); Ellen Jorgensen, Brooklyn, NY (US); German Sabio, Brooklyn, NY (US); Arjeta Markasevic, Brooklyn, NY (US); Noah Dworakowski, Brooklyn, NY (US)

(21) Appl. No.: 17/615,562

(22) PCT Filed: Jun. 1, 2020
(86) PCT No.: PCT/US20/35619

§ 371 Date: Nov. 30, 2021

Related U.S. Application Data
(60) Provisional application No. 62/854,363, filed on May 30, 2019.
(60) Provisional application No. 62/854,366, filed on May 30, 2019.
(60) Provisional application No. 62/970,920, filed on Feb. 6, 2020.
(60) Provisional application No. 62/972,367, filed on Feb. 10, 2020.

(51) Int. Cl. C12N 15/10; A23D 9/007; A23L 21/25; A23L 7/10; C12N 3/00; C12Q 1/6851

(52) U.S. Cl. CPC C12N 15/1065; A23D 9/007; A23L 21/25; A23L 7/10; C12N 3/00; C12Q 1/6851; A23V 2002/00; C12Q 1/689

(57) ABSTRACT

Described herein is biological barcodes that can be associated with physical articles for use in functioning as a unique identifier.

### PRIORITY

This application claims priority to U.S. Provisional Application Nos. 62/854,363 filed May 30, 2019, 62/854,366 filed May 30, 2019, and 62/972,367 filed Feb. 10, 2020, and are herein incorporated by reference in their entirety. This application also claims priority to U.S. Provisional Application entitled “Method of Tracking and Tracing Goods using Isothermal Amplification” filed Feb. 6, 2020.

### FIELD OF THE INVENTION

The invention disclosed herein relates generally to the field of tracing articles through the use of biologically-based identifiable macromolecules.

### BACKGROUND OF INVENTION

Traceability is an important aspect of the product supply chain. Transparency and control are critical to business success, and failure to achieve these goals can have extreme negative consequences. Counterfeit retail goods are a billion dollar industry, resulting in consumer fraud and hundreds of thousands of dollars in lost tax revenue. In 2016, counterfeit and pirated goods amounted to as much as 3.3% of world trade (OECD/EUIPO 2019, Trends in Trade in Counterfeit and Pirated Goods, Illicit Trade, OECD Publishing, Paris, https://doi.org/10.1787/g2g9f533-en.). In the agricultural sphere, adulterated or contaminated food affects both businesses and consumers. For companies, the average direct cost of one food recall is $10 million (“Recall Execution Effectiveness: Collaborative Approaches to Improving Consumer Safety and Confidence” 2010 Deloitte). Fines and indirect costs, including loss of business due to impact on brand reputation, can range significantly higher and can reach into the millions of dollars.

Consumers, distributors, retailers, and regulators all rely on methods of determining the origin and purity of various products. However, conventional technologies for tracking and tracing product lots are often easily duplicated and counterfeited. Newer technologies such as the use of artificial intelligence and block chain are ultimately rooted in the ability to tag reliably a product and have similar downsides. Standard barcoding is normally printed on packaging, which renders it useless for tagging products prior to packaging at early points in the supply chain. Traditional barcodes are easily counterfeited and can be separated from the product itself by changing the packaging.

Recently, a number of biotechnology-based solutions for tracking and tracing goods through supply chains have been proposed, with encoded nucleic acids such as DNA as the tagging agent (WO 2016/114808 A1, WO 2013/170009 A1, U.S. Pat. No. 10,302,614, US 2014/0220576 A1). However, nucleic acids are known to undergo hydrolysis, oxidation, and alkylation unless kept at well-controlled storage conditions, ideally dry and at low temperatures (“Protection and Deprotection of DNA—High-Temperature Stability of Nucleic Acid Barcodes for Polymer Labeling” Angew. Chem. Int. Ed. 2013, 52, 4269-4272). This adds significant limitations to their use in tracking and tracing. Although the double-stranded configuration of the DNA helix is stable under ideal conditions for long periods, it is unlikely to survive the harsh conditions found in many supply chains, particularly the problematic “first mile” from original source (farm, mine, etc.) to aggregation facilities, and initial processing that may involve conventional sterilization techniques such as autoclaving.

To increase its stability, DNA has been both chemically modified and combined with various substances with the aim of protecting it including Magnetic nanoparticles (“Combining Data Longevity with High Storage Capacity—Layer-by-Layer DNA Encapsulated in Magnetic Nanoparticles”, Advanced Functional Materials, 29, 28, (2019), glass (“Protection and Deprotection of DNA—High-Temperature Stability of Nucleic Acid Barcodes for Polymer Labeling” Angew. Chem. Int. Ed. 2013, 52, 4269-4272), silk (“DNA preservation in silk” Biomater Sci. 2017 Jun. 27; 5(7): 1279-1292.), and gelatin (“A self-monitored fluorescence DNA anti-counterfeiting system based on silica coated SYBR Green I/DNA gelatin nanoparticles”, Journal of Materials Chemistry 2017 vol 5, issue 24, p5939-5948). Unfortunately, the more effective the chemical and physical DNA protection strategies are, the less likely they are to be compatible with food use or to be safe for human or animal ingestion. In addition, such solutions may result in the nucleic acid being packaged in particles that are expensive or technically challenging to make, and may be expensive, time-consuming, or not scalable. Current tests for the detection of analytes, whether it is for individual molecules (inorganic and/or organic) or whole organisms, require complex settings, specialized instruments, and trained personnel, not to mention a long turnaround time. This impedes widespread adoption in different industries and their use in real life scenarios.

### SUMMARY OF INVENTION

The solution offered by the invention described herein leverages the durability, safety, and microscopic size of microorganisms, such as, but not limited to, a bacteria, virus, fungus, archaea, or algae to carry one or more biological barcodes that serve as a unique identifier to a physical article that is associated with the biological barcode. The present invention preserves a biological barcode through rigorous environmental conditions of certain supply chains. The biological barcode can be designed to allow for easy biological barcode readout via multiple analytical methodologies. Thus, allowing for a simpler, faster, and cost effective solution for tracking physical articles through a supply chain or other process comprising a transfer(s) of possession using microorganisms that are suitable for use in food and agriculture as well as electronics, industrial parts, gemstones, and labels.

The invention pertains to a system of biological barcodes that have a combination of conserved and barcode regions that allow for analyzing on a variety of detection/readout platforms with the use of set of primers or probes or crRNAs able to detect multiple biological barcodes with distinct barcode regions.

One aspect of this invention is nucleic acid biological barcodes and the method of use of said nucleic acid barcodes to identify physical articles to which the nucleic acid barcodes are added thereto or thereon or associated therewith. A nucleic acid biological barcode can be contained within a microorganism, such as a bacterial spore. Specifically, bacterial spores are genetically modified to carry the nucleic acid biological barcode in its genome or display it on its surface. In some embodiments, bacterial spores are genetically modified by inserting a nucleic acid biological barcode directly into the genome or by introduction an extrachromosomal element comprising the nucleic acid barcodes. The spore is then applied to the commercial article (e.g. by misting or spraying of bacterial spores comprising the nucleic acid barcodes onto the good), or associated with the product (e.g., by affixing a label that has bacterial spores comprises the nucleic acid barcodes incorporated therein).

A second aspect of the present disclosure include microorganisms, such as spores, comprising one or more recombinant biological barcodes and a genome modified to render inoperable one or more genes that are needed for spore germination or for production of an essential metabolite. As such, the microorganism can be non-germinating and/or auxotrophic. The microorganism can be, for example, Bacillus, Clostridium, and Saccharomyces. The microorganism can be a species selected from Bacillus subtilis, Bacillus cereus, Bacillus thuringiensis, Clostridium difficile, Clostridium perfringens, and Saccharomyces cerevisiae. The microorganism can be a spore.

A third aspect of the present disclosure includes a biological barcode comprising a nucleic acid sequence that comprises one or more conserved regions and one or more barcode regions, wherein each region has a different sequence. The biological barcode can have a configuration making a single configuration suitable for a variety of detection systems. Such biological barcodes can be located within a microorganism, such as a spore.

Another aspect of the disclosure is a system for identification of biological barcodes comprising a first biological barcode for associating with a first physical article and a second biological barcode for associating with a second physical article, wherein at least one conserved region of the first biological barcode has nucleic acid sequence that is the same as a nucleic acid sequence of a corresponding conserved region of the second biological barcode, and wherein at least one barcode region of the first biological barcode has a nucleic acid sequence that is different from a nucleic acid sequence of each barcode region of the second biological barcode. Such biological barcodes can be located within a microorganism, such as a spore. The system further comprises primers suitable for use with one or more detection systems.

A fourth aspect of the disclosure is a method of detecting a biological barcode associated with a physical article to identify the presence of the biological barcode or quantifying the amount of the biological barcode comprising extracting the biological barcode from the physical article or a portion thereof or from a label associated therewith and detecting the biological barcode. The method can further comprise determining the amount of barcode present in the physical article or a portion thereof.

A fourth aspect of the disclosure is a label configured to be affixed to a surface, such as the surface of a physical article, comprising a biological barcode and optionally a fluorescent indicator.

### DESCRIPTION OF FIGURES

FIGS. 1A-1F. Schematic depiction of various biological barcode embodiments of the present disclosure comprising a barcode region (e.g., barcode regions 1 and 2) and one or more conserved regions (e.g., conserved regions 1, 2, 3, 4, 5, and 6), a spacer.

FIGS. 2A and 2B Schematic depictions of a biological barcode and a set of qPCR primers. Depicts the binding of a biological barcode by the primers and probe(s) anneal to the indicated region. The direction of the arrow indicates if a primer is a forward primer (>, Primer 1, Primer 1.1., Primer 3) or reverse primer (<, Primer 2, Primer 4, and Primer 4.1). A probe is configured to bind to a barcode region of the biological barcode and is conjugated with a fluorophore (open circle) and a quencher (closed circle). Primer 1.1 and Primer 4.1 anneal to the nucleotides of genomic DNA adjacent to the first and sixth conserved region, respectively. In a variation for detecting the barcode using CRISPR based technologies, a crRNA is designed to bind to the same region bound by the probe used in qPCR.

FIGS. 3A and 3B Schematic depictions of a biological barcode and a set of LAMP primers. F, forward primer; B, backward primer; FIPa: 3 ‘-end of forward inner primer; FIPb: 5’-end of forward inner primer; BIPa: 3′ end of backward inner primer; BIPb: 5′-end of backward inner primer; LF: loop forward primer; LB: loop backward primer. F.1 and B.1 1 anneal to the nucleotides of genomic DNA adjacent to the first and sixth conserved region and can be used instead of primers F and B.

FIGS. 4A-4B. Detection of biological barcodes by using NGS. A) Primer 1 anneals to conserved region 1 and has an overhang comprising a sequencing primer site, a sample index, and the P5 sequence (adapter) required for binding to the flow cell. Primer 6 anneals to conserved region 6 and has an overhang comprising a sequencing primer site, a sample index, and the P7 sequence (adapter) required for binding to the flow cell. Primer 1.1 and Primer 6.1 anneal to nucleotides of genomic DNA adjacent to the first and sixth conserved region and can be used instead of primers 1 and 6. B) Quantitative detection of biological barcodes by using 2 sets of NGS primers, wherein Primer 1 and 6 contain an overhang comprising unique molecular identifiers (UMIs) and sequencing primer sites. UMIs are random nucleotides (4-8 base pairs long, NNNN). Primer SPS.1 and Primer SPS.6 were used for a second round of amplification and anneal to the sequencing primer sites of Primer 1 and Primer 6. Primer SPS.1 and SPS.6 comprise overhangs which consist of the P5 (forward) or P7 (reverse) adapter and an index required for attachment to the flow cell (P5/P7) and sample identification (indices).

FIG. 5 Outlines the synthesis of the biological barcode shown in FIG. 1E by using two rounds of PCR and subsequent insertion of the biological barcode into the genome of an organism.

FIG. 6. Depicts the results from the detection of N biological barcodes by qPCR and a single fluorophore. To detect barcode regions of N biological barcodes N probes conjugated to the same fluorophore are used wherein each fluorophore is added to the reaction mix at different and defined amounts resulting in predictable and measurable differences in the amplitude of the signal.

FIGS. 7A-7C. Determination of the detection sensitivity for different types of foods and liquids, in this case, rice, palm oil, and water, using qPCR. 10-fold dilutions were prepared starting from a stock of 1×10⁸ spores/mL and different concentrations of biological barcode-carrying spores were sprayed on rice (1×10⁵) and mixed with palm oil (5×10⁵), and water (5×10⁵). Genomic DNA was isolated from the tagged samples, specifically; 1 g of rice, 200 μL or palm oil, or 200 μL of water and the biological barcode was detected by qPCR using Taqman probes. Dashed line: Determined concentration of biological barcode in the sample; solid line: theoretical concentration of biological barcode in the sample.

FIG. 8. Detection of a biological barcode in honey. A plasmid with a biological barcode insert was transformed into B. subtilis to generate a “living device”. 5 μL of the spore preparation was added to 5 mL of honey and mixed by stirring. DNA was extracted from 1:1 diluted honey (in distilled water) and the DNA was then analyzed for the presence of the biological barcode using PCR with primers designed to specifically identify and read the biological barcode. Lane 1: Molecular Weight Marker, Lane 2: Untagged Honey, Lane 3: Tagged Honey.

FIGS. 9A-9B. Detection of two biological barcodes in water, rice, and palm oil. A) An aqueous stock of 1×10⁹ spores/mL was prepared and 1 mL was sprayed on 10 g of rice and mixed with 250 μl in 2.5 mL of palm oil. DNA was extracted in quintuplicate from 2 g of rice and 200 μL of palm oil and analyzed using qPCR. B) Two batches of water, palm oil, or rice containing different molecular barcodes (batch 1 contains biological barcode 1.1 and batch 2 contains biological barcode 3.1) were mixed in various ratios (batch1:batch2=100:0, 99:1, 90:10, 75:25, 50:50, and 0:100). DNA was extracted from each mixture ratio and analyzed by qPCR.

FIGS. 10A-10D. Results from stability study of biological barcodes inserted in the genome of non-germinating auxotroph spores compared to naked DNA. A) 70° C. water, B) 100° C. water, C) UV light, and D) autoclaving.

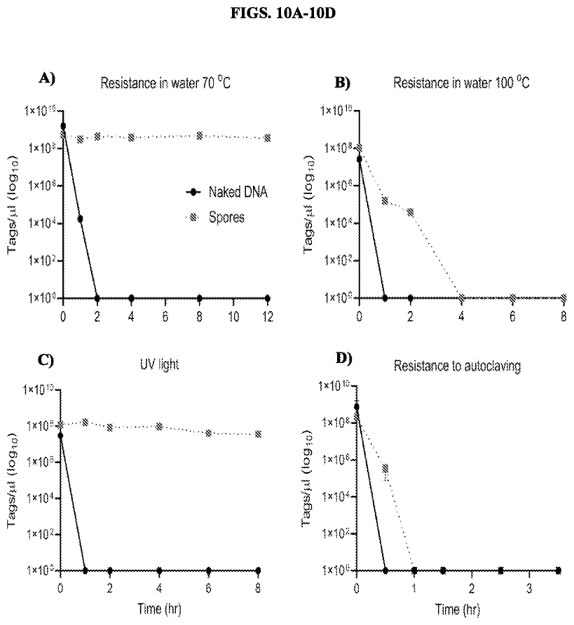

### DETAILED DESCRIPTION OF THE INVENTION

The present disclosure is directed to biological barcodes that can be associated with physical articles and function as unique identifier such as to identify commercial goods with which the biological barcodes are associated. A biological barcode is a biomolecule or combination of biomolecules such as DNA sequences, RNA sequences, proteins, peptides, hormones, metabolites, lipids, carbohydrates, oligosaccharides, or sugars. In use, a biological barcode would not normally be present in the physical article to which it is associated thus it can serve as a unique identifier of the physical article. Detection of the biological barcodes can be by any process commonly used by those versed in biotechnology, including but not limited to chemical, fluorescent and colorimetric assays (e.g. the Miller assay for beta-galactosidase activity, glucose detection with colored test strips), DNA or RNA detection methods (e.g. polymerase chain reaction (PCR), loop-mediated isothermal amplification, CRISPR-based technologies, immuno-PCR), DNA and/or RNA sequencing technologies (e.g. 16s sequencing, whole genome sequencing) antibody-based assays, or a combination thereof.

A recombinant microorganism can be the carrier of the biological barcode such as by integrating it into the genome of the microorganism, introducing a plasmid comprising the biological barcode into microorganism, or attaching the biological barcode to an exterior surface of the microorganism. The microorganism can be vegetative cells or engineered to be auxotrophic and/or in an irreversibly dormant or non-reproducing state.

The microorganism can be any spore forming organism. Microorganism can be a bacterium in a dormant state, e.g., a bacterial spore/endospore. In other embodiments, the microorganism is a fungal spore. Suitable carriers of the biological barcode can be selected from the group consisting of Bacillus, Clostridium, and Saccharomyces or more specifically, selected from the group consisting of Bacillus subtilis, Bacillus cereus, Bacillus thuringiensis, Clostridium difficile, Clostridium perfringens, and Saccharomyces cerevisiae.

In some embodiments, the microorganism has a combination of biological barcode types, for example, one or more nucleic acid biological barcodes integrated into the genome or contained on a plasmid therein and one or more protein or peptide biological barcodes attached to the surface. The protein may be expressed by the microorganism or attached to the exterior surface via a wet chemistry process, such as during manufacturing.

### Nucleic Acid Biological Barcodes

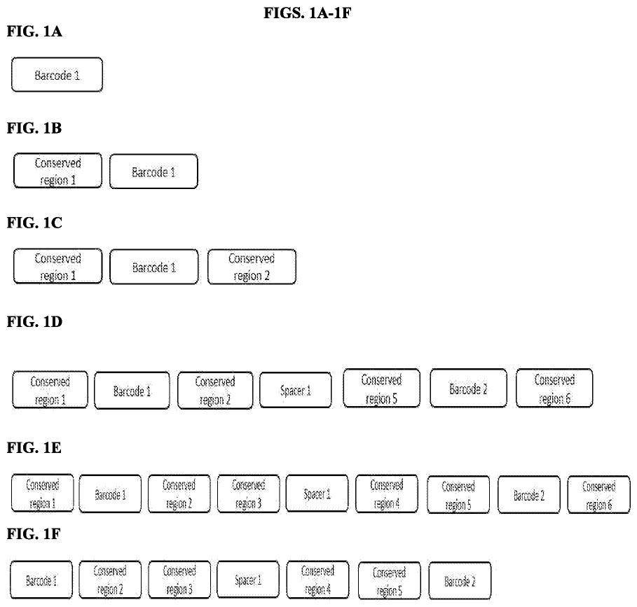

In some embodiments, a biological barcode comprises a single stranded or double stranded nucleic acid sequence. Exemplary nucleic acid biological barcodes of the present invention are shown in FIGS. 1A to 1F. A biological barcode can comprise one or more conserved regions and one or more barcode regions, wherein each region has a different sequence from the other regions within the biological barcode or differs from each other by 3 or more nucleotides or by at least 2%. In embodiments, each conserved region can be 10 to 50 or 12 to 40 or 15 to 25 nucleotides in length, and each barcode region can be 10 to 50 or 10 to 20 or 12 to 40 or 15 to 25 nucleotides in length. In embodiments, each conserved region or barcode region can have a GC content between 45% and 70% and an annealing temperature (Tm) between 50° C. and 70° C.

In an embodiment, a biological barcode consists of a barcode region (FIG. 1A), such as a series 10 to 50 nucleotides. In other embodiments, as depicted in FIG. 1B, the biological barcode comprises from a 5′/3′ end to a 3′/5′ end, a conserved region 1 and barcode region 1. As depicted in FIG. 1C, the biological barcode comprises from a 5′/3′ end to a 3′/5′ end, a conserved region 1, barcode region 1, and conserved region 2. As depicted in FIG. 1D, the biological barcode comprises from a 5′/3′ end to a 3′/5′ end, conserved region 1, barcode region 1, and conserved region 2, optionally, a spacer, conserved region 5, barcode region 2, and conserved region 6. As depicted in FIG. 1E, the biological barcode comprises from a 5′/3′ end to a 3′/5′ end, conserved region 1, barcode region 1, and conserved region 2, conserved region 3, optionally, a spacer, conserved region 4, conserved region 5, barcode region 2, and conserved region 6. As depicted in FIG. 1F, the biological barcode comprises from a 5′/3′ end to a 3′/5′ end, barcode region 1, and conserved region 2, conserved region 3, optionally, a spacer, conserved region 4, conserved region 5, and barcode region 2.

Regions within biological barcode can be spaced by 0 to 100 nucleotides. For example, with reference to FIG. 1E, conserved region 1 and barcode region 1 are spaced by 0 to 100 nucleotides, barcode 1 and conserved region 3 by 20 to 80 nucleotides, barcode 1 and barcode 2 by 120 to 200 nucleotides, barcode 2 and conserved region 4 by 20 to 80 nucleotides, barcode 2 and conserved region 6 by 0 to 100 nucleotides, and conserved region 3 and conserved region 4 by 0 to 100 nucleotides.

The biological barcodes shown in FIGS. 1A to 1E are suitable for use on NGS, qPCR and any CRISPR based technology. The biological barcode shown in FIG. 1E is suitable for detection with LAMP as well as NGS, qPCR and any CRISPR based technology. In some embodiments, a conserved region can consist of 15-25 nucleotides. In some embodiments, a barcode region can consist of 10-50 or 12-40 nucleotides. In an embodiment such as that depicted in FIG. 1E, the conserved region 2 and the conserved region 5, when present, consists of 10-50 or 12-40 nucleotides; the 1, 3, 4, and 6 conserved regions, when present, consists of 15-25; the spacer, when present, consists of 1-40 nucleotides; and each barcode region consists of 10-50 or 12-40 nucleotides. Table 1 provides an example of the parameters for a biological barcode like that shown in FIG. 1D.

TABLE 1

| Region | Length (nt) | Tm (° C.) | GC (%) |
|---|---|---|---|
| Conserved Region 1 | 15-25 | 50-70 | 45-70 |
| Barcode 1 | 12-40 | 50-70 | 45-70 |
| Conserved Region 2 | 12-40 | 50-70 | 45-70 |
| Conserved Region 3 | 15-25 | 50-70 | 45-70 |
| Spacer 1 | 0-40 | — | — |
| Conserved Region 4 | 15-25 | 50-70 | 45-70 |
| Conserved Region 5 | 12-40 | 50-70 | 45-70 |
| Barcode 2 | 12-40 | 50-70 | 45-70 |
| Conserved Region 6 | 15-25 | 50-70 | 45-70 |

### Microorganism Carrier

As mentioned above, a microorganism or cell can comprise a biological barcode. A microorganism can comprise 1, 2, 3, 4, 5, 6, 7, or more biological barcodes. The biological barcode can be configured to be incorporated into the microorganism so that it is not expressed by the microorganism. For example, the biological barcode integrated into the organism, genome or otherwise, does not comprise a promoter. In some embodiments, the biological barcode does not encode a gene or does not confer any fitness advantage.

The microorganism can be engineered to be non-germinating or nominally germinating and/or auxotrophic. For example, a bacterial or yeast spore of the present invention can be engineered to render inoperable genes that are critical to reproduction. In further or alternative embodiments, microorganism can be engineered to render inoperable genes encoding proteins required for essential functions or for the synthesis of essential metabolites, such as amino acids, vitamins, coenzyme synthesis, or other metabolites essential for nutrient uptake, thereby generating an auxotrophic strain to prevent growth in the absence of exogenous supply of such compounds, and hence from growing in the wild. For example, in some embodiments, the genome is modified not to express at least one of sleB, cwlD, and cwlJ or any combination thereof, specifically the combinations selected from sleB and cwlD; sleB and cwlJ; cwlD and cwlJ; and sleB, cwlD, and cwlJ. Genomes can further be modified not to express one or more of the group selected from gerD, all or individual genes of the gerA operon, gerAA, gerAB, all or individual genes of the gerB operon, gerC, all or individual genes of the gerK operon, gerP, gerT, gerM, gerQ, gerE, ypeB, pdaA, cotH, cotG, cotB, cotE, cotT, spoVAC, spoVAD, spoVAE, and sscA. Other gene encoding proteins required for germination, including germinant nutrient receptor or cell wall lytic enzymes, can also be knocked out to arrive at a microorganism carrier for a biological barcode.

The biological barcodes can be integrated into the genome at the site of one or more of the genes that are critical for reproduction or for essential metabolic functions. The insertion of the biological barcode at the such sites can result in disruption of the synthesis of the one or more genes and/or loss of function of the one or more essential genes. In some embodiments, insertion of the biological barcode results in deletion of the entire gene or by deletion of one or more exons in the case of eukaryotes.

In order to ensure that primers adapted to detect the biological barcode are not cross-reactive with sites within a sample and specific for the intended target, the nucleic acid sequence of the biological barcode or target regions therein (e.g., a conserved region or a barcode region) are not present or sufficiently distinct from those in the wild-type microorganism. For example, a barcode region and/or a conserved region, when present, each consist of a series of detectable N nucleotides that are not present in the wild-type microorganism or any other region of the biological barcode. In addition, to further mitigate cross-reactivity, the barcode region can differ by more than 3, 4, or 5 nucleotides from a series of N nucleotides in the wild-type spore and any other barcode or conserved region of the biological barcode. In embodiments, the barcode region consists of a series of N nucleotides that differ by more than 2%, 3%, 4,%, 5%, 7%, or 10% from a series of N nucleotides in the wild-type spore and any other conserved or barcode region of the biological barcode.

As mentioned above, microorganisms can comprise one or more recombinant amino acid-based biological barcode, wherein at least one of the one or more recombinant biological barcodes are located on the exterior surface of the microorganism and/or within the microorganism. Examples of amino acid biological barcodes include an enzyme, antibody, aptamer, fluorescent protein, receptor for a ligand, and antigen.

Spores as the microorganism carrier can be a stable means of storing and tracking a biological barcode, such as along a supply chain. The spores can have less than 5% degradation after storing for 3, 6, 12, 18, or 24 months under storage conditions comprising standard ambient temperature and pressure and humidity less than 50%. The spores can have less than 20% degradation after storing for 3, 6, 12, 18, or 24 months under environmental conditions comprising a temperature within −30° C. to 50° C., standard ambient pressure, and humidity less than 50%.

### Biological Barcode System

Another aspect of the disclosure is a system of a different biological barcodes or microorganisms comprising different biological barcodes as described herein. Within such systems, the conserved regions are conserved across the system of different biological barcodes, whereas the barcode regions are unique. Stated another way, at least one conserved region of the first biological barcode has nucleic acid sequence that is the same as a nucleic acid sequence of a corresponding conserved region of the second biological barcode and wherein at least one barcode region of the first biological barcode has a nucleic acid sequence that is different from a nucleic acid sequence of each barcode region of the second biological barcode. With such system, a universal primer can be used to analyze all the biological barcodes within the system. The system avoids the need to have a custom primer for each biological barcode within the system.

Accordingly, the system can comprise a plurality of comprise a plurality of different biological barcodes or microorganisms comprising different biological barcodes as described herein and a primer comprising a sequence that anneals with a conserved region from all the biological barcodes within the system. In some embodiments, for example, the system comprise a first forward primer comprising a sequence that anneals with the conserved region 1 from the multiple biological barcodes within the system and a second reverse primer comprising a sequence that anneals with the conserved region 2 from the multiple biological barcodes within the system. In further embodiments, a second reverse primer comprising a sequence that anneals with conserved region 2 from both the first biological barcode and the second biological barcode. At least one barcode region within each of the different biological barcodes is unique to that biological barcode.

In embodiments where the biological barcode is inserted into the genome or plasmid of a microorganism, the location of the biological barcode within the genome or plasmid can also serve as a unique identifier associated with the physical article(s) to which it is associated. Such identifier can be detected by designing a primer that targets a series of N nucleotides within the genome of the microorganism or plasmid near the insertion site of the biological barcode. For example, a sequence of the primer can comprise or consist of a sequence that anneals with a series of N nucleotides within a 1-100 nucleotide region of genomic DNA immediately upstream or downstream of the biological barcode, wherein N can be 1 to 40.

The system can be designed to utilize fluorescence as a means to identify and quantify the biological barcode. The system can comprise a probe (e.g., a molecular beacon) comprising a sequence that anneals a barcode region and a quencher and a fluorophore.

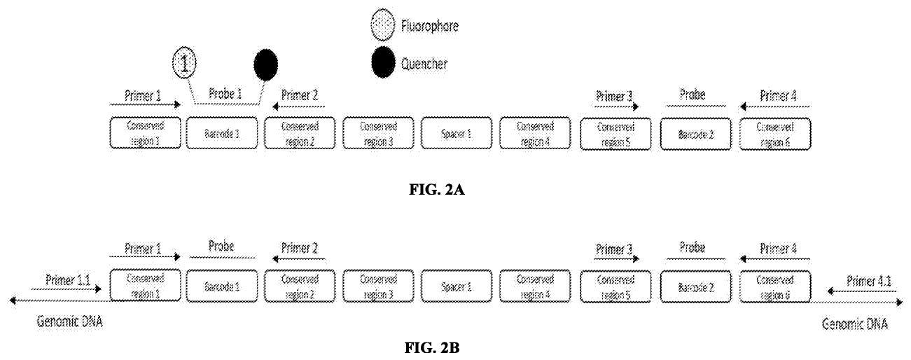

In other embodiments, the probe is a part of a set of primers suitable for qPCR as a means to identify and quantify the biological barcode. With the set of primers, more than one barcode region can be detected in a single reaction by using different barcode-specific probes in combination with universal primers, which bind to conserved regions of the biological barcode and/or genomic regions of the microorganism adjacent the conserved region. For example, a specific barcode region within a biological barcode can be detected using a primer:probe which binds to any region of the barcode region while the corresponding PCR primer pair is universal and binds to the conserved regions flanking the barcode region or the genomic region flanking biological barcode, wherein the amplicon generated by the flanking primers is 70 to 200 base pairs (bp) in length. In one embodiment, as illustrated in FIG. 2A, a set of primers can comprise the forward primer (primer 1) having a nucleic acid sequence that binds to conserved region 1 and the reverse primer (primer 2) having a nucleic acid sequence that binds to conserved region 2 (primer 2) or any region within the biological barcode or genome which lies within 0 to 200 bp from the 3′ end of the probe designed to bind to the barcode, wherein the forward and the reverse primers are universal and can be used in conjunction with any probe which are barcode specific. In a further embodiment, the reverse primer binds to conserved region 6 (primer 4) and the forward primer (primer 3) binds to any region within the biological barcode which lies within 0 to 200 bp from the 3′ end of the probe designed to bind to the barcode region, wherein the forward and the reverse primers are universal and can be used in conjunction with a barcode specific probe. In some embodiments, primer 1 and/or primer 4 bind directly to the genome or extrachromosomal element of the organism (primer 1.1. and primer 4.1. in FIG. 2B) thus allowing differentiation between identical biological barcodes which are integrated at different locations within the genome or extrachromosomal elements.

As shown in Example 3, a single type of fluorophore can be used on the different probes to detect the presence of different barcode regions by adding the probes to a test sample at different concentrations. Applying such technique, 1, 2, 3, 4, 5, or 6 fluorescent channels are used to detect 1, 2, 3, 4, 5, 6, 7, 8, 9, or 10 different biological barcodes in each channel by using defined amounts of each probe allowing the detection of 1 to 60 barcodes in a single reaction tube.

In embodiments, the system can be designed to utilize loop-mediated isothermal amplification (LAMP) as a means to identify and quantify the biological barcode. LAMP is a single tube, one-step amplification reaction that amplifies a target DNA sequence with high sensitivity and specificity under isothermal conditions (about 60-65° C.) using either two or three sets of primers and a polymerase with high strand displacement activity in addition to a replication activity. Typically 4 different primers are used to amplify 6 distinct regions on the target sequence, in this case the biological barcode. An additional pair of “loop primers” can further accelerate the reaction.

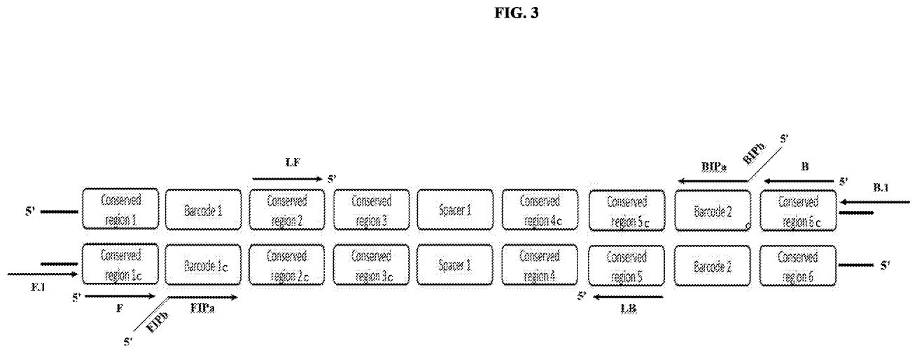

Accordingly, the system can further comprise a LAMP set of primers having four amplification primers (F, forward inner primer (FIP), backward inner primer (BIP), and B) and two loop primers (loop forward primer (LF) and loop backward primer (LB)) designed based on six regions in the biological barcode shown in FIG. 3A, wherein FIP and BIP comprise overhangs (FIPb and BIPb) which only bind upon a first round of amplification by FIPa and BIPa thereby resulting in the formation of a loop in the amplified product. The primers bind to the following regions of the biological barcode: F binds to conserved region 1, the 3′ end of FIP (FIPa) binds to barcode 1 and upon amplification the 5′-end of FIP (FIPb) binds to conserved region 3, the 3′ end of BIP (BIPa) binds to barcode 2 and upon amplification the 5′ end of BIP (BIPb) binds to conserved region 4, B binds to conserved region 6, LF binds to conserved region 2 only upon amplification by FIP, and LB binds to conserved region 5 only upon amplification by BIP, and wherein FIPa and BIPa control the specificity of the reaction.

In embodiments wherein the biological barcode is carried by the microorganism and the biological barcode is detected using LAMP, the set of primers and techniques as described above are the same with a further optional modification. Specifically, primer F and/or primer B can bind directly to the genome or extrachromosomal element of said organism (designated primer F.1. and primer B.1. in FIG. 3B) thus allowing differentiation between identical biological barcodes which are integrated at different locations within the organism's genome or extrachromosomal elements. Specificity of the set of primers can be controlled by replacing the entire nucleotide sequence of barcode region 1 and/or 2 or by replacing at least 3, at least 4, at least 5, or at least 6 nucleotides in the barcode region 1 and/or 2. In one embodiment, two specific barcodes within a biological barcode can be detected by using FIP and BIP primers which bind specifically to any region of the barcode region while the additional LAMP PCR primers (F, B, LB, and LF) are universal and bind to the conserved regions of all biological barcodes in the system, or alternatively the genomic region or extrachromosomal sequence adjacent to the biological barcode insertion site.

In some embodiments, the system can be designed to utilize NGS as a means to identify and quantify the biological barcode. NGS, also known as massive parallel sequencing, is a high-throughput sequencing method using the following general steps: First, DNA sequencing libraries are generated by clonal amplification by PCR in vitro. Second, the DNA is sequenced by synthesis, such that the DNA sequence is determined by the addition of nucleotides to the complementary strand rather than through chain-termination chemistry. Third, the spatially segregated, amplified DNA templates are sequenced simultaneously in a massively parallel fashion without the requirement for a physical separation step. While these steps are followed in most NGS platforms, each utilizes a different strategy. NGS platforms include those of Illumina, Ion Torrent, Minion, and PacBio. In a preferred embodiment, the NGS system platform utilized in the present invention is that of Illumina.

In some embodiments, the NGS sequencing format applied for detecting one or more biological barcodes is a single-end sequencing format (“sequencing only from one end of a sequencing library”) or a paired-end sequencing format (“sequencing from both ends of a sequencing library”), wherein the process of detecting the one or more biological barcodes is qualitative or quantitative. In some embodiments, the biological barcodes are dual-indexed, wherein indices are used during DNA sequence analysis to identify biological barcodes and are usually six base pairs long and allow up to 96 different biological barcodes to be run together.

As system for use with NGS can comprise a set of primers comprising forward and reverse primers comprise one or more elements selected from the group consisting of P5 adapter, P7 adapter, index, primer specific binding site, fluorophore, quencher dye, unique nucleotide identifier having a length between 15-40 nucleotides. The primer specific binding site (Read1) of the forward primer can comprise a sequence that anneals to a conserved region upstream of a barcode region. The primer specific binding site (Read 2) of the reverse primer can comprise a sequence that anneals to a conserved region downstream of a barcode region. In an embodiment, the NGS primers comprise from 5′ to 3′ the following elements: Forward: P5-Index1-Read1-TSP-F and Reverse: P7-Index2-Read2-TSP-R.

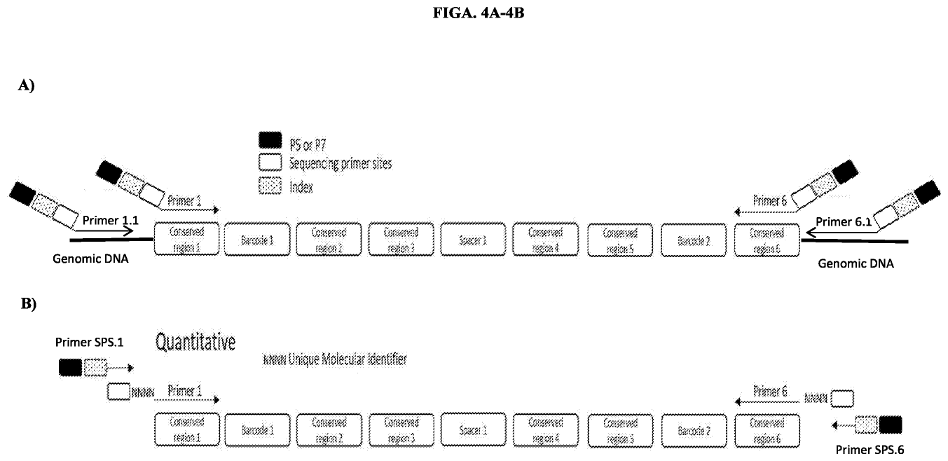

With reference to FIGS. 4A and 4B, a biological barcode of this invention can be detected using NGS by performing the following steps: (1) Reduced cycle amplification: Biological barcodes are recovered from the physical article and sequenced for primer binding (Read1 or Read2), indices (Index 1 or Index 2), and terminal sequences (P5 (forward) and P7 (reverse)) are added by PCR using tailed, target-specific primers (TSP-F and TSP-R). The resulting products are indexed, P5 and P7 tagged amplicons which are then further amplified, such as by about 25 rounds of PCR using generic P5 and P7 adapter primers creating thereby an indexed, P5 and P7 tagged library. The resulting library is then tagged to a flow cell that is coated with P5 and P7 probes and clonally amplified. (2) At the end of clonal amplification, all of the reverse strands are washed off the flow cell, leaving only forward strands. Primers attach to the forward strands and a polymerase adds fluorescently tagged nucleotides to the DNA strand. Only one base is added per round. Each flow cell is sequenced. (3) Data Analysis: Samples are demultiplexed based on indexes inserted in the amplicons and optionally unique molecular identifiers included in the NGS primers are used to quantify the amount of the barcodes in each sample.

A plurality of barcode regions within the same biological barcode, e.g., barcode 1 and barcode 2, can be sequenced using a single-end or paired-end NGS sequencing. For paired-end sequencing, the target-specific portions of the P5-containing NGS primer can bind to conserved region 1 and/or genomic nucleotides upstream and adjacent conserved region 1 (or upstream and adjacent barcode region 1, if no conserved region 1 present). Similarly, the P7-containing NGS primer binds to conserved region 6 and/or genomic nucleotides downstream and adjacent conserved region 6 (or upstream and adjacent barcode region 2, if no conserved region 6 present). The NGS sequencing format is a paired-end format, wherein the resulting NGS amplicon for sequencing has the following order of sequence elements: P5-Read1 Primer binding site-Index1-Barcode 1-Barcode2-Read2 primer binding site—Index2—P7. For single-end sequencing, only a P5-containing NGS primer needs to be used.

With paired-end sequencing, it is also an option for only one barcode of a plurality of barcodes within the same biological barcode to be detected. By way of example, the target-specific portions of the P5-containing NGS primers bind to conserved region 1 and the P7-containing NGS primer binds to any a region downstream of barcode 1 but upstream of barcode 2 and which fulfills the requirements to generate an amplicon of an appropriate length. NGS primers can be similarly designed for only detecting barcode 2.

### Methods of Using

Other aspects of the present invention are methods of detecting a biological barcode as described herein and associated with a physical article to identify the presence of the biological barcode or quantifying the amount of the biological barcode. The nature of the physical article to which the biological barcode is applied or mixed could be, but is not limited to, crops, oils, seeds, foods, packaged goods, precious stones, or any other material or item in any state, either solid or liquid. The methods can comprise extracting the biological barcode form a surface to which it is applied or a fluid in which it is mixed. The extraction process can comprise, for example, rinsing or swabbing an aliquot or defined area of a physical article with a solvent that will cause release of the biological barcode or its microorganism carrier from the physical article and into the solvent. The extraction process can also comprise recovering the biological barcode from within the microorganism so that it can be accessible for detection. Suitable solvents can include water or aqueous solutions containing guanidinium salts, nucleic acid stabilizing agents, acids, bases (e.g., sodium hydroxide), and/or detergents. In some embodiments, barcodes can be extracted from the microorganisms by using mechanical disruption and/or by using physical and/or chemical disruption. For example, the microorganism with biological barcode can be recovered by immersing the physical article in lysis buffer containing guanidinium salts and mixed with zirconia beads of different sizes for mechanical disruption. In other embodiments, the biological barcode or its microorganism can be released from the physical article into the solvent by immersing the physical article in a basic solution or acidic solution and heated to temperatures of 80° C. or above for a period of time. In some embodiments, solvents can be those that are suitable for use in a sequencing instrument, such as water. The method can further comprise adding primer(s) and/or probe(s) as described herein to the extract.

A NGS method for qualitative analysis of the biological barcode of FIG. 1E, for example, can comprise adding two primers to the extract, wherein the first primer anneals with at least a portion of the conserved region 1 and the second primer anneals with at least a portion of the conserved region 6, wherein the first primer comprises an overhang comprising a sequencing primer site, an index, and a P5 adapter and the second primer comprises an overhang comprising a sequencing primer site, an index, and a P7 adapter.

A NGS method for qualitative and quantitative analysis of the biological barcode comprising (1) adding two primers to the extract, wherein the first primer anneals with at least a portion of the conserved region 1 and the second primer anneals with at least a portion of the conserved region 6, wherein each primer has an overhang comprising a sequencing primer site and 2 to 20 random nucleotides and running 2 cycles of amplification; and (2) adding a second set of primers to the resulting amplification reaction, wherein the third primer comprises an overhang comprising an index and P5 adapter and anneals to the sequencing primer site of the first primer, and the fourth primer comprises an overhang comprising an index and P7 adapter and anneals to the sequencing primer site of the second primer and running 20 to 50 cycles of amplification.

For a method using a molecular beacon to detect the presence and amount of a biological barcode can comprise adding the molecular beacon to the extract wherein the beacon comprises a sequence that anneals with a barcode region of the biological barcode; and measuring an amount of fluorescence from the extract. In order to detect dilution or alteration of a physical article, the amount of measured fluorescence at one or more wavelengths in the extract can be compared to the amount of fluorescence of an extract obtained at another time or at another stage in a supply chain.

For a method using a qPCR sequencing to analyze the quality and quantity of a biological barcode, the method can comprise for each barcode region within a biological barcode to be detected, adding a probe specific to a barcode region, a forward primer, and reverse primer to the extract and measuring an amount of fluorescence from the extract. The forward primer and the reverse primer are specific to conserved regions flanking the barcode region. If multiple barcodes, the probes can have same or different fluorophores (e.g. 6-carboxyfluorescein or tetrachlorofluorescein) that fluoresce at same or different wavelengths. In order to detect dilution or alteration of a physical article, the amount of measured fluorescence at one or more wavelengths in the extract can be compared to the amount of fluorescence of an extract obtained at another time or at another stage in a supply chain.

A method of using LAMP to analyze the quality and quantity of a biological barcode can comprise adding a LAMP set of primers, namely, four amplification primers (F, forward inner primer (FIP), backward inner primer (BIP), and B) and two loop primers (loop forward primer (LF) and loop backward primer (LB)) designed based on six regions in the biological barcode shown in FIG. 3A, wherein FIP and BIP comprise overhangs (FIPb and BIPb) which only bind upon a first round of amplification by FIPa and BIPa to form a loop for amplifying the biological barcode. In some embodiments, amplification is conducted at a single temperature, for example at a temperature of 55° C., 56° C., 57° C., 58° C., 59° C., 60° C., 61° C., 62° C., 63° C., 64° C., 65° C., 66° C., 67° C., 68° C., 69° C., or 70° C., with 65° C. being preferred, for a time period of 5 to 300 minutes, wherein the number of rounds of amplification and generated copies of the target increases with increasing time duration of the run. The amplification product is detected via photometry, measuring the turbidity caused by magnesium pyrophosphate precipitate in solution as a byproduct of amplification or by fluorescence using intercalating dyes such as SYBR green. Dye molecules intercalate or directly label the DNA, and in turn can be correlated with the number of copies initially present. In a variation, changes in the color of the solution are detected as a function of changes in pH in the LAMP reaction. Hence, LAMP can also be quantitative.

### Method of Making a Carrier Microorganism

Further aspects of the present invention include a method of making a recombinant microorganism as described herein. The method can comprise producing a modified microorganism by inactivating one or more genes required for germination or essential metabolism, such as those listed herein; and inserting one or more biological barcodes as described herein in to the modified microorganism. One or more biological barcodes can be integrated into the genome of an organism using genome engineering methods and/or systems including but not limited to homologous recombination, the lambda red system, the Cre loxP system, or CRISPR-based technologies.

To remove or reduce any vegetative microorganisms in the modified microorganism, the method can further comprise exposing the modified microorganism to conditions that would be fatal to vegetative microorganisms, such as heat (such as between 50° C. to 85° C.), extreme pH, UV radiation, or an enzymatic treatment. To isolate only the microorganism with the inactivated genes, the method can comprise screening the modified microorganism by culturing in the presence of an antibiotic, wherein the modified microorganism has an antibiotic resistant gene in place of or disrupting the one or more genes required for germination. In further embodiments, to display a biological barcode on the surface of the microorganism, the method can comprising attaching a biological barcode to the surface of the microorganism or inserting a recombinant gene to express a biological barcode. Such biological barcodes are not naturally expressed by the microorganism. Such biological barcodes can be a peptide, enzyme, antibody, receptor, antigen, glycosylated protein having gene regulated sequence of saccharides, or an aptamer. Combinations of such biological barcodes are also contemplated. Each biological barcode as well as the combination can serve as an identifier.

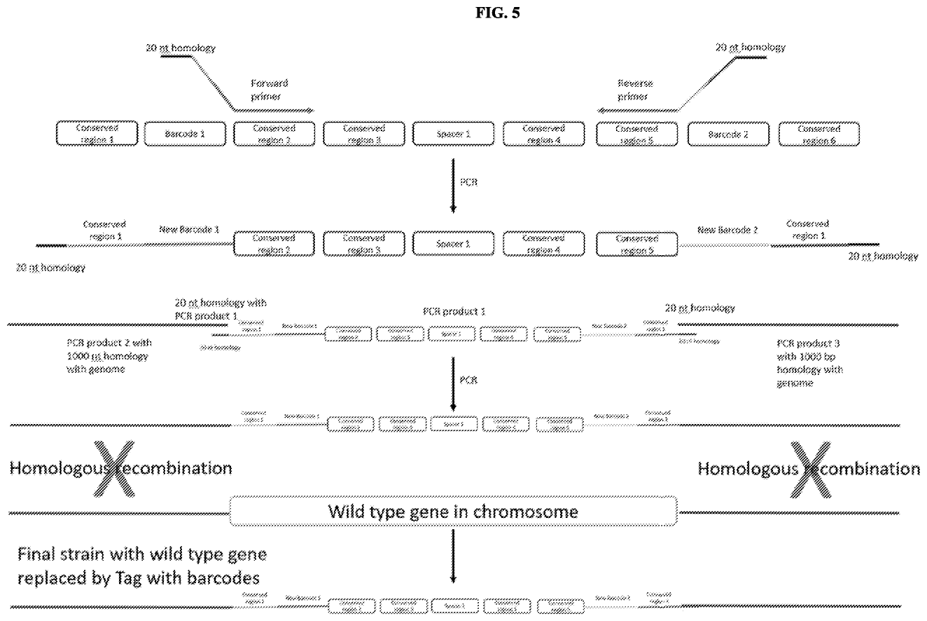

By way of example, a biological barcode vector for insertion to an microorganism can be prepared by the following method as depicted in FIG. 5: Step 1: A forward primer binding conserved region 2 and a reverse primer binding conserved region 5 are designed with overhangs at the 3′ end, wherein the forward primer comprises an overhang consisting of a barcode region 1, conserved region 1, and 20 nucleotides of homology to the region where the biological barcode will be integrated into the genome and the reverse primer comprises an overhang consisting of a barcode region 2, conserved region 6, and 20 nucleotides of homology to the region where the biological barcode will be integrated into the genome. Step 2: The generated biological barcode (PCR Product 1) is used in an overlapping PCR (“overlap extension PCR”) together with PCR products (PCR Product 2 and 3), wherein PCR Product 2 and 3 contain at least 1000 nucleotides of homology at the 5′ end at each side of the integration site and 20 nucleotides of homology at the 3′ end with the newly generated biological barcode. Step 3: The resulting PCR product (“biological barcode vector”) is inserted into a microorganism through homologous recombination, generating a living device with a biological barcode at the desired location.

Another aspect of the present invention is a physical article to which the biological barcode or microorganism carrying the same as described herein is associated therewith (such as by affixing thereto), incorporated therein, or applied thereon. In this way, the biological barcode is associated with physical articles moving through one or more supply chains or any other process comprising transfers of possession and/or location. Non-limiting examples of physical articles include foods, food-grade oils, honey, maple syrup, agricultural products, label glue, cannabis, electronics, consumer goods, pharmaceuticals, biologic test samples, gemstones and minerals.

In some embodiments, the suspended biological barcodes or microorganism carriers can be directly added into the product, such as mixed into a liquid. In yet another embodiment, biological barcodes or microorganism carriers can be added as a dry suspension. In some embodiments, biological barcodes or microorganisms are suspended in a carrier, which can then be applied such as by spraying, brushing, or dipping onto the physical articles to coat at least a portion thereof.

The carrier can be a polymer solution can be a glue and in particular, water soluble glue or wax. The carrier can be water, polysaccharides, polyethylene glycol, polyglycerols, agarose, agar, polish, resins, polyacrylamides, polyvinylpyrrolidinone, polyoxazoline, biofilms, or wax of any nature. The carrier, such as wax or the like, can reduce degradation rate of the biological barcodes or microorganisms.

In some embodiments, the biological barcode or microorganism after being applied to the physical thing (such as with a carrier) is covered with a protective layer that reduces the rate of degradation. The protective layer can be a wax coating or polymeric coating.

Another aspect of the present invention is a label to be affixed to a physical article or container or packaging of the physical article. A label can comprise one or more biological barcodes as described herein or microorganism carrying biological barcodes as described herein, and optionally a fluorescent indicator. The label can comprise one or more layers. In an embodiment, the label comprises a paper layer with the biological barcode or microorganism carrying the same applied thereto. The label can further comprise a fluorescent indicator. In some embodiments, the fluorescent indicator is located on the same layer as the biological barcode or a second layer of the label. In some embodiments, at least one of the biological barcodes is selected from a carbohydrate or sugar (e.g., glucose), aptamer, an enzyme, an antibody, receptor, and antigen. The biological barcodes or microorganisms can be dispersed within a glue. The glue can be used to hold the biological barcode or microorganism to the label, such as to the paper layer. In some embodiments, the glue can be used to affix the label to the physical article or to bind the layers of the label together. In embodiments, the glue is water soluble.

In some embodiments, a probiotic biological barcode can be a specified blend of microorganisms carrying biological barcode(s). The combination of certain species being an identifier and/or the relative concentrations of certain species.

### EXAMPLES

### Example 1

A genetically modified strain of Bacillus subtilis carrying the gene for red fluorescent protein was engineered using standard genetic engineering methods. The gene for red fluorescent protein (RFP) was cloned into plasmid PHY300PLK (Takara Biosciences). The RFP gene was obtained from plasmid pSB1C3 containing the BioBrick BBa j04450 by using it as a template for the polymerase chain reaction (PCR). The PCR product, which contained the RFP gene, was digested with the restriction endonucleases Pstl and EcoRI, after which the enzymes were inactivated by heat (80° C. for 20 min.). Plasmid PHY300PLK was digested with the restriction endonucleases Pstl and EcoRI and the enzymes similarly inactivated. The PCR product containing the RFP gene and the digested pHY300PLK plasmid were mixed together and ligated. The ligation was transformed into a K12-derived laboratory strain of E. coli and plated on LB agar medium containing Ampicillin. Plates were incubated overnight at 37° C. and produced red E. coli colonies that were found to contain pCAR01, a plasmid derived from PHY300PLK but containing the RFP gene. It was confirmed that E. coli containing pCAR01 (and showing red color) were resistant to both Amp and Tet, as would be expected from proper construction of the pCAR01 plasmid by plating on LB containing either ampicillin or tetracycline.

The plasmid pCAR01 was purified from the genetically engineered E. coli using standard alkaline lysis followed by DNA capture on silica resin columns (New England Biolabs Monarch plasmid miniprep kit). Bacillus subtilis strain 168 was made competent to facilitate transformation with pCAR01 using a procedure adapted from Molecular Biological Methods for Bacillus (1990) C. M. Harwood and S. M. Cutting, Wiley Publications.

The following solutions were prepared:

### T Base

| Reagents | Amount [g/L] | Cone [mM] |
|---|---|---|
| (NH₄)₂SO₄ | 2 | 15 |
| K₂HPO₄ · 3H₂O | 18.3 | 80 |
| KH₂PO₄ | 6 | 44 |
| Trisodium Citrate · 2H₂O | 1 | 3.6 |

### SpC Medium (20 mL)

Made fresh the day of use from the following reagents:

| Reagents | Amount [mL] |
|---|---|
| TBase | 20 |
| 50% (w/v) Glucose | 0.2 |
| 1.2% (w/v) MgSO₄ · 3H₂O | 0.3 |
| 10% (w/v) Bacto Yeast Extract | 0.4 |
| 1% (w/v) Casamino Acids | 0.5 |
| 2 mg/mL L-Tryptophan | 0.2 |

### SpII Medium (200 mL)

Made fresh the day of use from the following reagents:

| Reagents | Amount [mL] |
|---|---|
| T Base | 200 |
| 50% (w/v) Glucose | 2 |
| 1.2% (w/v) MgSO₄ · 3H₂O | 14 |
| 10% (w/v) Bacto Yeast Extract | 2 |
| 1% (w/v) Casamino Acids | 2 |
| 0.1 MCaCl₂ | 1 |
| 2 mg/mL L-Tryptophan | 2 |

SpII Me+ EGTA

200 mL SPII (without CaCl₂), with 4 mL EGTA (0.1M, pH 8). Medium was frozen at −20 C in single use (˜0.5 mL) aliquots.

Competent B. subtilis cells were prepared according to the following protocol: Day 1: Streak out the strain to be made competent onto LB agar as a large patch and incubate overnight at 30° C. Day 2: Scrape the cell growth off the plate and use to inoculate 20 mL of fresh, pre-warmed SpC medium. OD600 should read close to 0.5. The culture was incubated at 37° C. with vigorous aeration and periodic OD600 readings were taken to assess cell growth. When growth stalled (no significant change in cell density for 20-30 minutes), 200 mL of pre-warmed SpII medium were inoculated with 2 mL of stationary-phase culture. Incubation was continued at 37° C. with slower aeration. After 90 minutes of incubation, the cells were pelleted by centrifugation at 8,000 g for 5 minutes at room temperature, the supernatant was decanted and saved. The pellet was resuspended in 18 mL of saved supernatant. 2 mL of sterile glycerol were added and mixed gently. 0.5 mL aliquots were prepared, rapidly frozen in LN2, dry ice/EtOH, or ice/isopropanol, and stored at −70° C.

Transformation of B. subtilis strain 168 with pCAR01 was achieved by rapidly thawing competent cells in a 37° C. water bath and immediately adding one volume SpII+ EGTA to thawed cells with gentle mixing. 100 μL of pCAR01 DNA solution containing about 600 ng DNA was added to 0.2 mL of these competent cells, after which they were incubated at 37° C. on a rotator for 60 minutes. Transformations were plated onto selective media (LB agar with tetracycline 50 μg/mL). Resulting B. subtilis colonies were amplified by inoculating LB media and incubating the inoculated LM media at 37° C., 200 rpm overnight. DNA was extracted from the resulting bacteria culture using a Zymo Quick-DNA™ Fungal/Bacterial Microprep Kit, and screened for the RFP gene by PCR. The presence of the pCAR01 plasmid was confirmed by using the NEB Monarch DNA miniprep kit with a preliminary step incubating the cells in 5 mg/mL lysozyme prior to addition of lysis buffer. A strain containing the RFP gene in pCAR01 was designated 168/pCAR01 and archived in 50% glycerol LB media stored at −80 C.

Spores were prepared from B. subtilis as follows. 4 mL of LB media was inoculated with B. subtilis and incubated overnight at 37° C. with shaking at 200 rpm. The next day the OD600 was measured and the culture diluted with LB media to an OD600 of 0.1 for a final volume of 10 mL and placed back in the incubator-shaker at 37° C., 200 rpm until the OD600 reached 0.8. The cells were pelleted by centrifugation at 13,000×g for 1 minute, washed once with PBS, and resuspended in 5 mL Difco Sporulation Media (DSM). The resuspended cells were incubated at 37° C. with shaking at 200 rpm for 24 h, after which they were treated with 5 mg/mL lysozyme for 1 h at room temperature and then washed 6 times with PBS. After the final wash they were resuspended in 2 mL PBS. The presence of spores was confirmed by microscopy.

### Example 2—Preparation of Bacillus subtilis 168 with Knockouts of gerD, cwlD, and SleB

Bacillus subtilis 168, the wild type strain (trpC2), was engineered to knockout genes gerD, cwlD, and sleB. Genes were interrupted with an antibiotic resistance cassette flanked by loxP sites. The antibiotic resistance cassettes used were kanamycin or erythromycin.

Individual trpC2 ΔgerD::erm, trpC2 ΔcwlD::kan and trpC2 ΔsleB::kan were obtained from Bacillus Genetic Stock Center. Bacillus subtilis 168 strains were grown and genomic DNA (gDNA) was extracted and used as template for a PCR using primers that bind approximately 1000 nucleotides upstream and downstream of the 5′ and 3′ ends of the antibiotic resistance cassettes. PCR products were gel purified and used to transform wild type strain Bacillus subtilis 168.

Briefly, the wild type strain was grown overnight in MC media and diluted 1:100 in competence media and grown to an OD600 of 0.8. 120 μl of culture grown in competence media were transformed with a minimum of 100 ng of PCR product. The entire volume of the transformation was plated on LB plates supplemented with erythromycin or kanamycin (depending on the strain) and incubated overnight at 37° C. Transformants were verified for loss of wild type gene by colony PCR using primers specific to each gene (gerD, cwlD, and SleB). To remove antibiotic resistance cassettes, transformants verified by colony PCR were grown overnight in 3 mL of MC media supplemented with the appropriate antibiotic. The culture was diluted 1:100 in competence media, grown until OD₆₀₀ of 0.8, and transformed with at least 100 ng of plasmid pDR244 encoding the Cre recombinase. Transformation was plated on LB plates with ampicillin and after overnight growth at 30° C., individual colonies were streaked at 42° C. for 16 hours to remove the plasmid. Correct loss of the antibiotic resistance cassette was verified by PCR as described above. This was repeated 3 times until removing all 3 genes in a single strain.

### Example 3—Sensitive and Specific Detection of a Biological Barcode Using LAMP Assay

In order to determine the specificity of the detection method, three samples were prepared: Sample 1 was a biological barcode comprising a series of nucleotides in accordance with the parameters described herein; Sample 2 was a plasmid carrying the gene encoding RFP, an engineered mutant form of red fluorescent protein from the coral Discosoma striata, and Sample 3 was wild-type genomic DNA isolated from B. subtilis. Each sample was analyzed with a LAMP primer designed to target the biological barcode of sample 1.

A positive result indicates that the biological barcode was present in the sample.

As shown in Table 2 below, the Samples 2 and 3 were negative for the presence of the biological barcode. And the primers showed no cross-reactivity (no fluorescent signal) with the genome of B. subtilis or with the plasmid carrying the gene encoding RFP. These results show that a biological barcode system has specificity.

TABLE 2

| | Sample 1 | Sample 2 | Sample 3 |
|---|---|---|---|
| Targets | Biological barcode | Plasmid with RFP | B. subtilis genome |
| Result | Positive—Primer | Negative—None | Negative—None |
| | binding detected | detected | detected |

To further evaluate the specificity of the LAMP-based barcode detection method, an increasing number of mutations (2, 3, 5, or 6 mutations) was incorporated into the 3 ‘-end region or 5’-end region of the forward inner primer (FIPa) and the backward inner primer (BIPa) which bind to barcode region 1 or barcode region 2, respectively. The mutated FIPa and BIPa primer were then used for the detection assay of a biological barcode. All other primers were conserved in the LAMP assay. As shown in Table 3, 2, 3, 4, and 5 mutations negatively impact primer binding.

TABLE 3

| # of Mutations | 0 | 3 | 2 | 3 | 2 |
|---|---|---|---|---|---|
| Mutated Primer | — | FIPa | BIPa | FIPb | BIPb |
| Result | Positive | Negative | Positive | Slightly | Slightly |
| | | | | Positive | Positive |
| # of Mutations | 0 | 5 | 6 | 5 | 6 |
| Mutated Primer | — | FIPa | BIPa | FIPb | BIPb |
| Result | Positive | Negative | Negative | Negative | Negative |

To measure the sensitivity of the LAMP assay, an aqueous solution containing a biological barcode was serially diluted, and each step of dilution (Table 4) was analyzed for the presence of the biological barcode by LAMP (65° C., 60 minutes, primers as outlined in FIG. 3A) and the amplification product was detected by fluorescence. As shown in Table 3, the assay is sensitive down to at least 100 copies since as few as 100 copies (5 fg) of a biological barcode in a sample were sufficient to generate a positive signal using the LAMP assay.

TABLE 4

| | Sample Concentration | | | | | | | |
|---|---|---|---|---|---|---|---|---|
| | 50 ng | 5 ng | 0.5 ng | 50 pg | 5 pg | 0.5 pg | 50 fg | 5 fg |
| Result | Positive | Positive | Positive | Positive | Positive | Positive | Positive | Slightly |
| | | | | | | | | Positive |

### Example 2—Detection of a Biological Barcode Using qPCR with a Generic Fluorophore

A single type of fluorophore can be used across multiples probes with different targets can be used to detect multiple biological barcodes in accordance with the present disclosure by qPCR. With this approach, each probe is added to the reaction mix at different and defined amounts resulting in predictable and measurable differences in the amplitude of the signal.

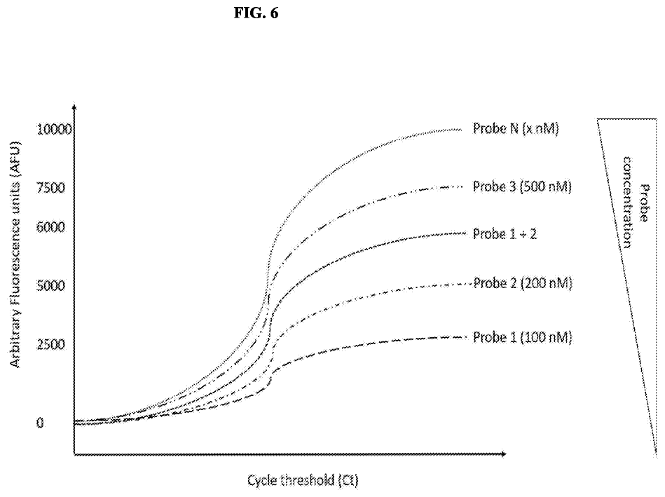

For example, Probe 1 is added at a final concentration of 100 nM which corresponds to a maximum fluorescence of 2,500 AFU, independent of the amount of molecular barcode present in the mixture, as the amount of available probe is exhausted. Probe 2 is added at a final concentration of 200 nM which corresponds to a maximum fluorescence of 5,000 AFU. Consequently, using primers 1 and 2, observing a maximum AFU of 2,500 indicates the presence of barcode 1, whereas a maximum AFU is 5,000 indicates the presence of barcode 2. Detection of a maximum AFU of 7,500 indicates that both barcodes is detected in the same sample (additive AFU) (FIG. 6).

### Example 3—Detection Limit of a Biological Barcode in Water, Rice, and Palm Oil Using qPCR

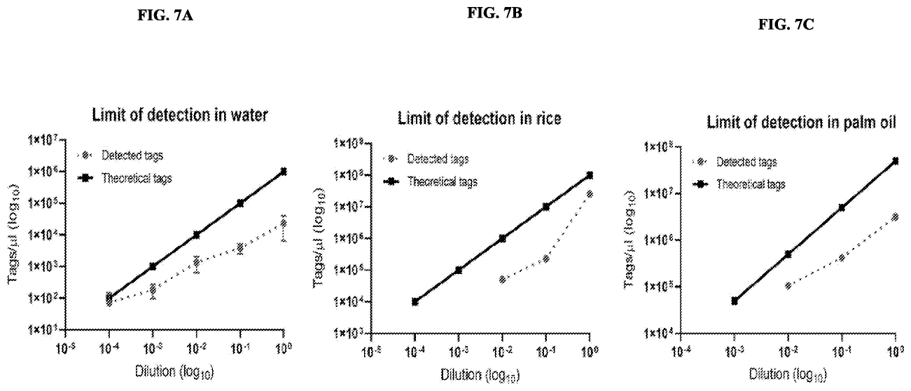

In order to determine the detection sensitivity for different products using qPCR, 10-fold dilutions were prepared starting from a stock of biological barcode-carrying spores as described herein (1×10⁸/mL) and different concentrations of such spores were added to rice (1×10⁵), palm oil (5×10⁵), and water (5×10⁵). Genomic DNA was isolated from the tagged samples using 1 g of rice, 200 μL of palm oil or 200 μL of water. The amount of barcode present in each sample was then detected using Taqman qPCR. The limit of detection of tagged spores across different products (water, palm oil, and rice) is similar under laboratory conditions, ranging from 1×10⁵ spores for rice and 5×10⁵ spores for palm oil (FIG. 7)

### Example 4—Detection of a Biological Barcode in Honey and Palm Oil

Honey: The biological barcode was validated for use in honey as follows. 5 μL spores were added to 5 mL honey and mixed thoroughly by stirring. To retrieve the biological barcode, the honey was diluted 1:1 with distilled water to improve flow, and DNA extracted from 200 μL using the Zymo Quick-DNA™ Fungal/Bacterial Microprep Kit. Honey without the biological barcode was also extracted and used as a control sample. The resulting extracted DNA was analyzed and the presence of the biological barcode confirmed via PCR with primers specific to the biological barcode. A PCR band was only obtained in the sample containing DNA extracted from tagged honey, while no signal was obtained when using DNA extracted from untagged honey (FIG. 7)

Palm oil: The biological barcode was validated for use in palm oil as follows. 0.5 μg of pCAR01 plasmid DNA was added to 5 mL palm oil and mixed by stirring (“tagged palm oil”). The presence of the tag was confirmed using LAMP. 1 μL of the tagged palm oil was added to a LAMP mixture containing 12.5 μL WarmStart® Colorimetric LAMP 2× Master Mix from NEB, 9 μl., nuclease-free water, and 2.5 μL of a master mix of the primers as described in Example 3. Similar to the results obtained for honey, a positive signal was only obtained in the sample containing tagged palm oil, while no signal was obtained when using untagged palm oil (Table 5).

TABLE 5

| | Untagged Control | Tagged Palm Oil | Positive Control |
|---|---|---|---|
| Result | Negative | Positive | Positive |

### Example 5—Detection of Two Biological Barcodes in Water, Rice, and Palm Oil

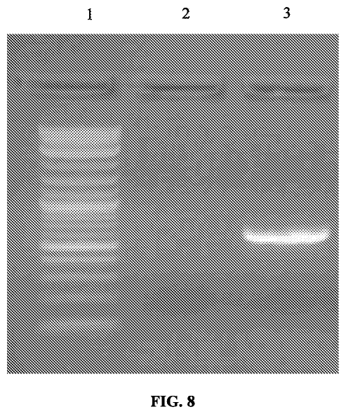

In order to assess tagging uniformity, a stock of 1×10⁹ tag-carrying spores/mL was prepared and 1 mL was added to 10 g of rice or 250 μL to 2.5 mL of palm oil and thoroughly mixed. DNA was extracted in quintuplicate from 2 g of rice or 200 μL of palm oil and analyzed using qPCR. As shown in FIG. 8A, biological barcodes were detected at similar levels in all samples.

In order to assess whether the living device tagging system can be used to determine when a product has been mixed with other lots of tagged product or diluted with untagged products, two batches of water, palm oil, or rice containing different molecular barcodes (batch 1 contains biological barcode 1.1 and batch 2 contains biological barcode 3.1) were mixed in various ratios (batch1:batch2=100:0, 99:1, 90:10, 75:25, 50:50, and 0:100). DNA was extracted from each mixture ratio of water, rice, or palm oil and analyzed by qPCR.

When comparing mixed samples against initial time point (100:0), estimations of the level of dilution and/or mixing can be determined (FIG. 8B).

### Example 6—Stability of Biological Barcodes Inserted into the Genetic Information of Spores

In order to test the stability of biological barcodes inserted into the genetic information of spores compared to naked nucleic acid barcodes, stock solutions of 1×10⁸ spores/mL with biological barcodes integrated into the genome or 1×10⁸ biological barcodes/mL in water (naked DNA) were continuously exposed to different conditions: 70° C. water, 100° C. water, UV light (254 nM), or autoclaving (121° C. and 15 psi). The number of biological barcodes per μL were detected using qPCR.

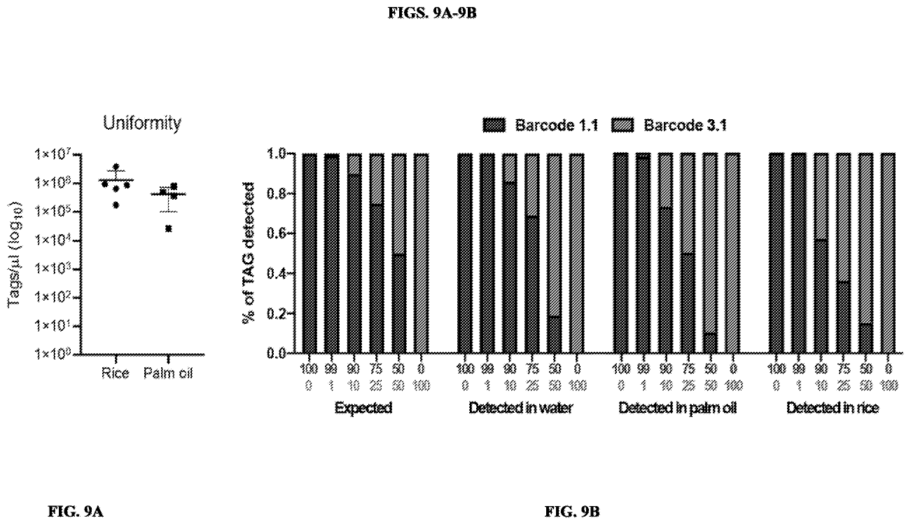

Under all tested conditions, spores outlasted naked DNA and biological barcodes could be detected after hours of continuous exposure to high temperatures or UV light (254 nm), and even after 30 min of autoclaving (121° C. and 15 psi) (FIG. 9). No signal could be detected for naked DNA under any of the tested conditions, highlighting the fragility of naked DNA.

### Example 7

A LAMP test identifying a container of palm oil was conducted. A barcode consisting of 0.5 ug DNA from a plasmid containing a unique DNA sequence derived from a gene from coral was added to 5 mL palm oil and mixed thoroughly. The presence of the barcode was confirmed using loop-mediated isothermal amplification (LAMP). 1 uL of the barcode-containing palm oil was added to a LAMP mixture containing 12.5 uL WarmStart® Colorimetric LAMP 2× Master Mix from NEB, 9 uL nuclease-free water, and 2.5 uL of a master mix of the following primers was used to obtain the following concentration of primers shown in Table 6:

TABLE 6

| | | 1X Conc. |
|---|---|---|
| Primer | Primer Sequence | (FINAL) |
| FIP | GCGGTCTGGGTACCTTCGTACCGGTCACGAG | 1.6 μM |
| | TTCGAAATCG (SEQ ID NO: 1) | |
| BIP | AGTTACCAAAGGTGGTCCGCTGGCTTTGGAA | 1.6 μM |
| | CCGTACTGGAA (SEQ ID NO: 2) | |
| F3 | GTTCGTATGGAAGGTTCCGT | 0.2 μM |
| | (SEQ ID NO: 3) | |
| B3 | CAGCCGGGTGTTTAACGT | 0.2 μM |
| | (SEQ ID NO: 4) | |
| LOOP F | CCACTTCCACTTCCAGCAGG | 0.4 μM |
| | (SEQ ID NO: 5) | |
| LOOP B | CGTTCGCTTGGGACATCCTGT | 0.4 μM |
| | (SEQ ID NO: 6) | |

The positive control and the palm oil containing the barcode turned yellow, indicating the presence of the barcode, whereas no barcode was detected in the negative control. The amount of DNA added was less than a microgram, and did not affect the color or taste of the palm oil in any way.

It is understood that the following examples and embodiments described herein are for illustrative purposes and that various modifications or changes in light thereof will be suggested to persons skilled in the art and are to be included within the spirit and purview of this application and scope of the appended claims.

All publications referred to herein are hereby incorporated by reference.

1. Isolated spores comprising one or more recombinant biological barcodes and a genome modified to render inoperable two or more genes that are needed for spore germination.

2. (canceled)

3. (canceled)

4. The spores of claim 1, wherein the spores are Bacillus, Clostridium, and Saccharomyces or wherein the spores are a species selected from Bacillus subtilis, Bacillus cereus, Bacillus thuringiensis, Clostridium difficile, Clostridium perfringens, and Saccharomyces cerevisiae.

5. The spores of claim 1, wherein the genome does not express sleB, cwlD, and cwlJ.

6. The spores of claim 1, wherein the genome does not express gerD.

7. The spore of claim 1, where the genome does not express a gene selected from the gerA operon, gerAA, gerAB, gerB operon, gerC, gerK operon, gerP, gerT, gerM, gerQ, gerE, ypeB, pdaA, cotH, cotG, cotB, cotE, cotT, spoVAC, spoVAD, spoVAE, and sscA or wherein the genome does not express a gene encoding a germinant nutrient receptor and/or a cell wall lytic enzyme.

8. The spores of claim 1, wherein at least one of the one or more recombinant biological barcodes is a nucleic acid sequence comprising one or more barcode regions, wherein the barcode region consists of a series of N nucleotides that are not present in the wild-type spore or any other region of the biological barcode and differs by three or more nucleotides from a series of N nucleotides in the wild-type spore or any other region of the biological barcode, wherein N is at least 12.

9. The spores of claim 8, wherein the biological barcode consists of 12-1000 nucleotides.

10.-19. (canceled)

20. The spore of claim 1, wherein degradation of the isolated spores is less than 5% after storing for 3, 6, 12, or 24 months under storage conditions comprising standard ambient temperature and pressure and humidity less than 50% or wherein degradation of the isolated spores is less than 20% after storing for 3, 6, 12, or 24 months under environmental conditions comprising a temperature within −30° C. to 50° C., standard ambient pressure, and humidity less than 50%.

21. (canceled)

22. The spore of claim 1, wherein the one or more recombinant biological barcode comprises one or more amino acid sequences, or wherein at least one of the one or more recombinant biological barcodes are located on the exterior surface of the spore or wherein at least one of the one or more recombinant biological barcodes is an enzyme, antibody, aptamer, fluorescent protein, receptor, or antigen.

23.-40. (canceled)

41. A system for identification of biological barcodes comprising
a first spore according to the spore of claim 1 and a second spore according to the spore of claim 1, wherein the first spore is associated with a first physical article and
the second spore is associated with a second physical article,
wherein the biological barcode of the first spore has a nucleic acid sequence that is different from a nucleic acid sequence of the biological barcode of the second spore.

42. The system of claim 41, wherein the first and second biological barcodes are suitable for detection with isothermal amplification NGS, qPCR and a CRISPR-based assay.

43. The system of claim 41, wherein each conserved region of the first biological barcode has nucleic acid sequence that is the same as a nucleic acid sequence of a corresponding conserved region of the second biological barcode.

44. (canceled)

45. (canceled)

46. (canceled)

47. (canceled)

48. The system of claim 41, wherein the spores are Bacillus, Clostridium, and Saccharomyces or wherein the spores are a species selected from Bacillus subtilis, Bacillus cereus, Bacillus thuringiensis, Clostridium difficile, Clostridium perfringens, and Saccharomyces cerevisiae.

49. The system of claim 41, wherein the genome does not express at least two of sleB, cwlD, and cwlJ.

50. The system of claim 41, wherein the genome does not express gerD.

51. The system of claim 41, where the genome does not express a gene selected from the gerA operon, gerAA, gerAB, gerB operon, gerC, gerK operon, gerP, gerT, gerM, gerQ, gerE, ypeB, pdaA, cotH, cotG, cotB, cotE, cotT, spoVAC, spoVAD, spoVAE, and sscA or wherein the genome does not express a gene encoding a germinant nutrient receptor and/or a cell wall lytic enzyme.

52.-66. (canceled)

67. A method of detecting a biological barcode associated with a physical article to identify the presence of the biological barcode or quantifying the amount of the biological barcode comprising extracting the biological barcode from the physical article or a portion thereof or from a label associated therewith, wherein the biological barcodes are contained within a spore according to claim 1.

68.-88. (canceled)

89. A method of making a recombinant spore comprising
producing modified spores by inactivating two or more genes required for spore germination or essential metabolic function; and
inserting one or more biological barcodes comprising nucleotide sequence of at least 10 base pairs in to the modified spores.

90.-92. (canceled)

93. A label configured to be affixed to a surface comprising a biological barcode and optionally a fluorescent indicator.

94.-98. (canceled)

99. The label of claim 93, comprising a glue within which the biological barcodes are dispersed, optionally wherein the glue is water soluble.
